SEQUENCE LISTING (4 sequences)

SEQ ID NO: 1 - DNA, 1470 bp, Corynebacterium glutamicum
  CDS (235)..(1266)
ctcactcccgcaggagcccaagaggctctgggcagccagatgggatggactggcatgctgtccgcactaaaagcgtggctggaatacggagtgaacctccgcgacgggttttataagcaataggcaatgtgtccatcacgatgtgtggcggattatgatccatgtaacaagaatgtgcagtttcacagaactgacaatcaacttattttgacctgacaaaaggagcgacgacacatggccacattcaaacaggccagaagcgctgcctggctgatcgcccccgccctcgtggtccttgcagtggtgatcggatatcccatcgtccgagcaatttggctatccttccaggccgacaaaggcctcgaccccaccaccggactcttcaccgacggtggcttcgcaggactagacaattacctctactggctcacccaacgatgcatgggttcagacggcaccatccgtacctgcccacccggcacactagccaccgacttctggccagcactacgcatcacgttgttcttcaccgtggttaccgtcggcttggaaactatcctcggcaccgccatggcactgatcatgaacaaagaattccgtggccgcgcacttgttcgcgcagcgattcttatcccttgggcaatccccaccgccgtcaccgcaaaactgtggcagttcatcttcgcaccacaaggcatcatcaactccatgtttggacttagtgtcagttggaccaccgatccgtgggcagctagagccgccgtcattcttgccgacgtctggaaaaccacaccattcatggcactgctgatcctcgccggtctgcaaatgatcccgaaggaaacctacgaagcagcccgcgtcgatggcgcaaccgcgtggcagcaattcaccaagatcaccctcccgctggtgcgcccagctttgatggtggcagtactcttccgcaccctcgatgcgctacgcatgtatgacctccccgtcatcatgatctccagctcctccaactcccccaccgctgttatctcccagctggttgtggaagacatgcgccaaaacaacttcaactccgcttccgccctttccacactgatcttcctgctgatcttcttcgtggcgttcatcatgatccgattcctcggcgcagatgtttcgggccaacgcggaataaagaaaaagaaactgggcggaaccaaggatgagaaacccaccgctaaggatgctgttgtaaaggccgattctgctgtgaaggaagccgctaagccatgactaaacgaacaaaaggactcatcctcaactacgccggagtggtgttcatcctcttctggggactagctcccttctactggatggttatcaccgcactgcgcgattccaagcacacctttgacaccaccccatggccaacgcacgtcaccttggataacttccgggacgcactggccaccgacaaaggcaacaacttcctcg

SEQ ID NO: 2 - protein, 344 aa, Corynebacterium glutamicum
MATFKQARSAAWLIAPALVVLAVVIGYPIVRAIWLSFQADKGLDPTTGLFTDGGFAGLDNYLYWLTQRCMGSDGTIRTCPPGTLATDFWPALRITLFFTVVTVGLETILGTAMALIMNKEFRGRALVRAAILIPWAIPTAVTAKLWQFIFAPQGIINSMFGLSVSWTTDPWAARAAVILADVWKTTPFMALLILAGLQMIPKETYEAARVDGATAWQQFTKITLPLVRPALMVAVLFRTLDALRMYDLPVIMISSSSNSPTAVISQLVVEDMRQNNFNSASALSTLIFLLIFFVAFIMIRFLGADVSGQRGIKKKKLGGTKDEKPTAKDAVVKADSAVKEAAKP

SEQ ID NO: 3 - DNA, 19 bp, Artificial Sequence
  Synthetic DNA
agcgattcttatcccttgg

SEQ ID NO: 4 - DNA, 19 bp, Artificial Sequence
  Synthetic DNA
agcaggaagatcagtgtgg

(19) United States
(12) Patent Application Publication
(10) Pub. No.: US 2002/0127661 A1
(43) Pub. Date: Sep. 12, 2002

(54) Nucleotide sequences coding for the sugA gene

(75) Inventors: Mike Farwick, Bielefeld (DE); Klaus Huthmacher, Gelnhausen (DE); Walter Pfefferle, Halle (DE); Thomas Hermann, Bielefeld (DE); Achim Marx, Bielefeld (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: DEGUSSA AG, Duesseldorf (DE)

(21) Appl. No.: 09/951,753

(22) Filed: Sep. 14, 2001

(30) Foreign Application Priority Data
Sep. 14, 2000 (DE) 100 45 485.2
Feb. 23, 2001 (DE) 101 08 839.6

(51) Int. Cl. C12P 13/04; C07H 21/04; C12N 1/21; C12N 15/74

(52) U.S. Cl. 435/106; 435/252.3; 435/320.1; 536/23.2

(57) ABSTRACT

The invention relates to an isolated polynucleotide containing a polynucleotide sequence selected from the group

(a) polynucleotide that is at least 70% identical to a polynucleotide coding for a polypeptide that contains the amino acid sequence of SEQ ID No. 2,

(b) polynucleotide coding for a polypeptide that contains an amino acid sequence that is at least 70% identical to the amino acid sequence of SEQ ID No. 2,

(c) polynucleotide that is complementary to the polynucleotides of a) or b), and

(d) polynucleotide containing at least at least 15 successive nucleotides of the polynucleotide sequence of a), b) or c),

and a process for the enzymatic production of L-amino acids using coryneform bacteria in which at least the sugA gene is present in attenuated form, and the use of polynucleotides that contain the sequences according to the invention as hybridization probes.

### BACKGROUND OF THE INVENTION

1. Field of the Invention

The present invention provides nucleotide sequences of coryneform bacteria coding for the sugA gene and a process for the enzymatic production of amino acids using bacteria in which the sugA gene is attenuated.

2. Description of the Background

L-amino acids, in particular L-lysine, are used in human medicine and in the pharmaceutical industry, in the foodstuffs industry and, most especially, in animal nutrition. It is known that amino acids can be produced by fermentation of strains of coryneform bacteria, in particular Corynebacterium glutamicum. On account of their great importance, efforts are constantly being made to improve the production processes of amino acids. Process improvements may involve fermentation technology measures such as, for example, stirring and provision of oxygen, or the composition of the nutrient media, such as, for example, the sugar concentration during the fermentation, or the working-up to the product form by, for example, ion exchange chromatography or the intrinsic performance properties of the microorganism itself.

In order to improve the performance properties of these microorganisms, methods involving mutagenesis, selection and mutant selection are employed. In this way, strains are obtained that are resistant to antimetabolites or are auxotrophic for regulatorily important metabolites, and that produce amino acids.

For some years methods of recombinant DNA technology have also been used to improve L-amino acid-producing strains of Corynebacterium, by amplifying individual amino acid biosynthesis genes and investigating the effect on amino acid production.

### SUMMARY OF THE INVENTION

It is an object of the present invention to provide nucleic acids which are useful for producing amino acids.

It is another object of the present invention to provide novel microorganisms which are useful for producing amino acids.

It is yet another object of the present invention to provide processes for producing amino acids.

The objects of the invention, and others, may be accomplished with an isolated polynucleotide from coryneform bacteria containing a polynucleotide sequence coding for the sugA gene, selected from the group

(a) a polynucleotide that is at least 70% identical to a polynucleotide coding for a polypeptide that contains the amino acid sequence of SEQ ID No. 2,

(b) a polynucleotide coding for a polypeptide that contains an amino acid sequence that is at least 70% identical to the amino acid sequence of SEQ ID No. 2,

(c) a polynucleotide that is complementary to the polynucleotides of (a) or (b), and

(d) a polynucleotide containing at least at least 15 successive nucleotides of the polynucleotide sequence of (a), (b), or (c),

where the polypeptide preferably having the activity of the sugar transport protein SugA.

The invention also provides the aforementioned polynucleotide, which is preferably a replicable DNA containing:

(i) the nucleotide sequence shown in SEQ ID No. 1, or

(ii) at least one sequence that corresponds to the sequence (i) within the region of degeneracy of the genetic code, or

(iii) at least one sequence that hybridizes with the sequences that are complementary to the sequences (i) or (ii), and, optionally,

(iv) functionally neutral sense mutations in (i).

The invention furthermore provides a replicable polynucleotide, in particular DNA, containing the nucleotide sequence as shown in SEQ ID No. 1;

a polynucleotide coding for a polypeptide that contains the amino acid sequence as shown in SEQ ID No. 2;

a vector containing parts of the polynucleotide according to the invention, but at least 15 successive nucleotides of the claimed sequence, and

coryneform bacteria in which the sugA gene is attenuated, in particular by an insertion or deletion.

The invention moreover provides polynucleotides that consist substantially of a polynucleotide sequence that can be obtained by screening by means of hybridization of a corresponding gene library of a coryneform bacterium that contains the complete gene or parts thereof, with a probe that contains the sequence of the polynucleotide of the invention according to SEQ ID No. 1 or a fragment thereof, and isolation of the aforementioned polynucleotide sequence.

The present invention also provides Coryneform bacteria, in which the sugA gene is attenuated.

The present invention also provides a process for the enzymatic production of an L-amino acid, comprising:

(a) fermentating coryneform bacteria producing the L-amino acid in a medium, wherein at least the sugA gene or a nucleotide sequence coding for the latter is attenuated in the bacteria,

(b) enriching the amount of the L-amino acid in the medium or in the cells of the bacteria, and

(c) isolating the L-amino acid.

The present invention also provides a process for identifying nucleic acids which code for the sugar transport protein sugA or that have a high degree of similarity to the sequence of the sugA gene, comprising:

contacting a sample with the isolated polynucleotide described above under conditions suitable for the polynucleotide to hybridize to other nucleic acids which code for the sugar transport protein sugA or that have a high degree of similarity to the sequence of the sugA gene.

A more complete appreciation of the invention and many of the attendant advantages thereof will be readily obtained as the same becomes better understood by reference to the following Figure in conjunction with the detailed description below.

### BRIEF DESCRIPTION OF THE FIGURES

FIG. 1: Map of the plasmid pCR2.1sugAint.

### DETAILED DESCRIPTION OF THE INVENTION

The abbreviations and acronyms used have the following meanings:

| | |
|---|---|
| KmR: | Kanamycin resistance gene |
| EcoRI: | Cleavage site of the restriction enzyme EcoRI |
| HindIII: | Cleavage site of the restriction enzyme HindIII |
| Sad: | Cleavage site of the restriction enzyme Sad |
| sugAint: | Internal fragment of the sugA gene |
| ColE1: | Replication origin of the plasmid ColE1 |

When L-lysine or lysine are mentioned hereinafter, this is understood to refer not only to the bases, but also to the salts, such as for example lysine monohydrochloride or lysine sulfate. In fact, when L-amino acids or amino acids in general are mentioned hereinafter, it is understood that this refers to one or more amino acids including their salts, selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-lysine is particularly preferred.

Polynucleotides that contain the sequences according to the invention are suitable as hybridization probes for RNA, cDNA and DNA in order to isolate nucleic acids or polynucleotides or genes in their full length that code for the sugar transport protein SugA, or to isolate such nucleic acids and/or polynucleotides or genes that have a high similarity to the sequence of the SugA gene. They are also suitable for incorporation in so-called “arrays”, “micro arrays” or “DNA chips” in order to detect and determine the corresponding polynucleotides.

Polynucleotides that contain the sequences according to the invention are furthermore suitable as primers with the aid of which, and by employing the polymerase chain reaction (PCR), DNA of genes can be produced that code for the sugar transport protein SugA.

Such oligonucleotides serving as probes or primers contain at least 25, 26, 27, 28, 29 or 30, preferably at least 20, 21, 22, 23 or 24, and most particularly preferably at least 15, 16, 17, 18 or 19 successive nucleotides. Also suitable are oligonucleotides with a length of at least 31, 32, 33, 34, 35, 36, 37, 38, 39 or 40, or at least 41,42, 43, 44, 45, 46, 47, 48, 49 or 50 nucleotides. Also optionally suitable are oligonucleotides with a length of at least 100, 150, 200, 250 or 300 nucleotides.

The term “isolated” denotes a material separated from its natural environment.

The term “polynucleotide” refers in general to polyribonucleotides and polydeoxyribonucleotides, which may be unmodified RNA or DNA or modified RNA or DNA.

The polynucleotides according to the invention include a polynucleotide according to SEQ ID No. 1 or a fragment produced therefrom, and also polynucleotides that are at least 70% to 80%, preferably at least 81% to 85%, particularly preferably at least 86% to 90%, and most particularly preferably at least 91%, 93%, 95%, 97% or 99% identical to the polynucleotide according to SEQ ID No. 1 or a fragment produced therefrom.

The term “polypeptides” is understood to mean peptides or proteins that contain two or more amino acids bound by peptide bonds.

The polypeptides according to the invention include a polypeptide according to SEQ ID No. 2, in particular those with the biological activity of the sugar transport protein SugA and also those that are at least 70% to 80%, preferably at least 81% to 85%, particularly preferably at least 86% to 90%, and most particularly preferably at least 91%, 93%, 95%, 97% or 99% identical to the polypeptide according to SEQ ID No. 2 and that have the aforementioned activity.

The invention furthermore provides a process for the enzymatic production of amino acids selected from the group L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine, using coryneform bacteria that in particular already produce amino acids and in which the nucleotide sequences coding for the sugA gene are attenuated, in particular switched off, or are expressed at a low level.

The term “attenuation” used in this context describes the reduction or switching off of the intracellular activity of one or more enzymes (proteins) in a microorganism that are coded by the corresponding DNA, by for example using a weak promoter or using a gene or allele that codes for a corresponding enzyme having a low activity or that inactivates the corresponding gene or enzyme (protein), and optionally combining these measures.

By such attenuation measures the activity or concentration of the corresponding protein is in general reduced to 0 to 75%, 0 to 50%, 0 to 25%, 0 to 10% or 0 to 5% of the activity or concentration of the wild-type protein, or the activity or concentration of the protein in the starting microorganism.

The microorganisms that are the subject of the present invention are able to produce amino acids from glucose, sucrose, lactose, fructose, maltose, molasses, starch, cellulose or from glycerol and ethanol. The microorganisms may be representatives of coryneform bacteria, in particular of the genus Corynebacterium. In the genus Corynebacterium there should in particular be mentioned the species Corynebacterium glutamicum, which is known to those skilled in the art for its ability to produce L-amino acids.

Suitable strains of the genus Corynebacterium, in particular of the species Corynebacterium glutamicum (C. glutamicum), are in particular the known wild type strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 und

Brevibacterium divaricatum ATCC14020

and mutants or strains obtained therefrom that produce L-amino acids.

The new sugA gene coding for the sugar transport protein SugA has been isolated from C. glutamicum.

In order to isolate the sugA gene or also other genes from C. glutamicum, a gene library of this microorganism is first of all incorporated in Escherichia coli (E. coli). The incorporation of gene libraries is described in generally known textbooks and manuals. As examples there may be mentioned the textbook by Winnacker: Gene and Klone, Eine Einführung in die Gentechnologie (Verlag Chemie, Weinheim, Germany, 1990) or the manual by Sambrook et al.: Molecular Cloning, A Laboratory Manual (Cold Spring Harbor Laboratory Press, 1989). A very well-known gene library is that of the E. coli K-12 strain W3110, which was incorporated by Kohara et al. (Cell 50, 495-508 (1987)) into λ vectors. Bathe et al. (Molecular and general genetics, 252:255-265, 1996) describe a gene library of C. glutamicum ATCC13032 that has been incorporated by means of the cosmid vector SuperCos I (Wahl et al., 1987, Proceedings of the National Academy of Sciences USA, 84:2160-2164) in the E. coli K-12 strain NM554 (Raleigh et al., 1988, Nucleic Acids Research 16:1563-1575).

Börmann et al. (Molecular Microbiology 6(3), 317-326) (1992)) in turn describe a gene library of C. glutamicum ATCC13032 using the cosmid pHC79 (Hohn and Collins, Gene 11, 291-298).

In order to produce a gene library of C. glutamicum in E. coli, there may also be used plasmids such as pBR322 (Bolivar, Life Sciences, 25, 807-818 (1979)) or pUC9 (Vieira et al., 1982, Gene, 19:259-268). Suitable hosts are in particular those E. coli strains that are restriction-defective and recombinant-defective such as for example the strain DH5αmcr, which has been described by Grant et al. (Proceedings of the National Academy of Sciences USA, 87 (1990) 4645-4649). The long DNA fragments cloned with the aid of cosmids or other λ vectors can in turn then be subcloned into common vectors suitable for the DNA sequencing and subsequently sequenced, as is described for example by Sanger et al. (Proceedings of the National Academy of Sciences of the United States of America, 74:5463-5467, 1977).

The DNA sequences obtained can then be investigated using known algorithms or sequence analysis programs, such as for example that of Staden (Nucleic Acids Research 14, 217-232(1986)), that of Marck (Nucleic Acids Research 16, 1829-1836 (1988)) or the GCG program of Butler (Methods of Biochemical Analysis 39, 74-97 (1998)).

The new DNA sequence of C. glutamicum coding for the sugA gene has been discovered, and as SEQ ID No. 1 is part of the present invention. The amino acid sequence of the corresponding protein was also derived from the existing DNA sequence using the afore-described methods. The resultant amino acid sequence of the sugA gene product is shown in SEQ ID No. 2.

Coding DNA sequences that result from SEQ ID No. 1 due to the degeneracy of the genetic code are likewise within the scope of the present invention. Similarly, DNA sequences that hybridize with SEQ ID No. 1 or parts of SEQ ID No. 1 are also part of the invention. In the art, conservative amino acid replacements, such as for example the replacement of glycine by alanine or of aspartic acid by glutamic acid, in proteins are furthermore known as sense mutations that do not lead to any basic change in the activity of the protein, i.e. are functionally neutral. It is furthermore known that changes at the N-end and/or C-end of a protein do not significantly impair their function or indeed may even stabilize their function. A person skilled in the art can find relevant information on this in, inter alia, Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks and manuals on genetics and molecular biology. Amino acid sequences that are obtained in a corresponding manner from SEQ ID No. 2 are likewise within the scope of the invention.

In the same way, DNA sequences that hybridize with SEQ ID No. 1 or parts of SEQ ID No. 1 are also within the scope of the present the invention. Finally, DNA sequences that are produced by the polymerase chain reaction (PCR) using primers resulting from SEQ ID No. 1, are also part of the invention. Such oligonucleotides typically have a length of at least 15 nucleotides.

A person skilled in the art can find information on the identification of DNA sequences by means of hybridization in, inter alia, the manual “The DIG System User's Guide for Filter Hybridization” published by Boehringer Mannheim GmbH (Mannheim, Germany, 1993) and in Liebl et al. (International Journal of Systematic Bacteriology 41: 255-260 (1991)). The hybridization takes place under strict conditions, in other words only hybrids are formed in which the probe and target sequence, i.e. the polynucleotides treated with the probe, are at least 70% identical. It is known that the strictness of the hybridization conditions including the washing step is influenced or determined by varying the buffer composition, temperature and the salt concentration. The hybridization reaction is preferably carried out under conditions that are relatively less strict compared to the wash steps (Hybaid Hybridisation Guide, Hybaid Limited, Teddington, UK, 1996).

For the hybridization reaction there may for example be used a 5' SSC buffer at a temperature of ca. 50° C.-68° C. In this connection probes can also hybridize with polynucleotides that are less than 70% identical to the probe sequence. Such hybrids are less stable and are removed by washing under stringent conditions. This may be achieved for example by reducing the salt concentration to 2× SSC and then if necessary to 0.5× SSC (The DIG System User's Guide for Filter Hybridization, Boehringer Mannheim, Mannheim, Germany, 1995), a temperature of ca. 50° C.-68° C. being established. It is also possible to reduce the salt concentration down to 0.1× SSC. By stepwise raising of the hybridization temperature in steps of ca. 1-2° C. from 50° C. to 68° C., polynucleotide fragments can be isolated that are for example at least 70% or at least 80% or even at least 90% to 95% identical to the sequence of the probe that is used. Further details relating to hybridization may be obtained in the form of so-called kits available on the market (e.g. DIG Easy Hyb from Roche Diagnostics GmbH, Mannheim, Germany, Catalogue No. 1603558).

The person skilled in the art can find details on the amplification of DNA sequences by means of the polymerase chain reaction (PCR) in, inter alia, the manual by Gait: Oligonucleotide Synthesis: A Practical Approach (IRL Press, Oxford, UK, 1984) and in Newton and Graham: PCR (Spektrum Akademischer Verlag, Heidelberg, Germany, 1994).

It has been found that coryneform bacteria after attenuation of the sugA gene produce amino acids in an improved way.

In order to achieve an attenuation, either the expression of the sugA gene or the catalytic properties of the enzyme protein may be reduced or switched off. Optionally, both measures may be combined.

The reduction of the gene expression may be achieved by suitable culture conditions or by genetic alteration (mutation) of the signal structures of the gene expression. Signal structures of the gene expression are for example repressor genes, activator genes, operators, promoters, attenuators, ribosome binding sites, the start codon and terminators. The person skilled in the art can obtain further information on this in for example patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Pátek et al. (Microbiology 142: 1297 (1996)), Vasicova et al. (Journal of Bacteriology 181: 6188 (1999)) and in known textbooks of genetics and molecular biology, such as for example the textbook by Knippers (“Molekylare Genetik”, 6^{th} Edition, Georg Thieme Verlag, Stuttgart, Germany, 1995) or the textbook by Winnacker (“Gene and Klone”, VCH Verlagsgesellschaft, Weinheim, Germany, 1990).

Mutations that lead to an alteration or reduction of the catalytic properties of enzyme proteins are known; as examples there may be mentioned the work of Qiu and Goodman (Journal of Biological Chemistry 272: 8611-8617 (1997)), Sugimoto et al. (Bioscience Biotechnology and Biochemistry 61: 1760-1762 (1997)) and Möckel (“Die Threonindehydratase aus Corynebacterium glutamicum: Aufhebung der allosterischen Regulation und Struktur des Enzyms”, and reports published by the Jülich Research Center, Jül-2906, ISSN09442952, Jülich, Germany, 1994). Overviews may be obtained from known textbooks on genetics and molecular biology, for example that of Hagemann (“Allgemeine Genetik”, Gustav Fischer Verlag, Stuttgart, 1986).

Mutations in the present context include transitions, transversions, insertions and deletions. Depending on the effect of the amino acid replacement on the enzyme activity, one talks either of missense mutations or nonsense mutations. Insertions or deletions of at least one base pair (bp) in a gene lead to frame shift mutations, following which false amino acids are incorporated or the translation terminates prematurely. Deletions of several codons typically lead to a complete cessation of enzyme activity. Details of the production of such mutations are well-known and may be obtained from known textbooks on genetics and molecular biology, such as for example the textbook by Knippers (“Molekylare Genetik”, 6^{th} Edition, Georg Thieme Verlag, Stuttgart, Germany, 1995), the textbook by Winnacker (“Gene and Klone”, VCH Verlagsgesellschaft, Weinheim, Germany, 1990) or the textbook by Hagemann (“Allgemeine Genetik”, Gustav Fischer Verlag, Stuttgart, 1986).

A conventional method of mutating genes of C. glutamicum is the method of gene disruption and gene replacement described by Schwarzer and Pühler (Bio/Technology 9, 84-87 (1991)).

In the method of gene disruption a central part of the coding region of the gene in question is cloned into a plasmid vector that can replicate in a host (typically E. coli), but not in C. glutamicum. Suitable vectors are for example pSUP301 (Simon et al., Bio/Technology 1, 784-791 (1983)), pK18mob or pK19mob (Schäfer et al., Gene 145, 69-73 (1994)), pK18mobsacB or pK19mobsacB (Jäger et al., Journal of Bacteriology 174: 5462-65 (1992)), pGEM-T (Promega Corporation, Madison, Wis., USA), pCR2.1-TOPO (Shuman (1994), Journal of Biological Chemistry 269:32678-84; U.S. Pat. No. 5,487,993), pCR®Blunt (Invitrogen, Groningen, Netherlands; Bernard et al., Journal of Molecular Biology, 234: 534-541 (1993)) or pEM1 (Schrumpf et al, 1991, Journal of Bacteriology 173:4510-4516). The plasmid vector that contains the central part of the coding region of the gene is then converted by conjugation or transformation into the desired strain of C. glutamicum. The method of conjugation is described for example by Schäfer et al. (Applied and Environmental Microbiology 60, 756-759 (1994)). Methods of transformation are described for example in Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a cross-over event, the coding region of the relevant gene is disrupted by the vector sequence and two incomplete alleles are obtained, missing respectively the 3′- and 5′-end. This method has been used for example by Fitzpatrick et al. (Applied Microbiology and Biotechnology 42, 575-580 (1994)) to switch off the recA gene of C. glutamicum.

In the gene replacement method a mutation, such as for example a deletion, insertion or base replacement, is produced in vitro in the gene that is of interest. The resultant allele is in turn cloned into a non-replicative vector for C. glutamicum, and this is then converted by transformation or conjugation into the desired host of C. glutamicum. After homologous recombination by means of a first cross-over event effecting integration, and an appropriate second cross-over event effecting an excision, the incorporation of the mutation or allele in the target gene or in the target sequence is achieved. This method has been used for example by Peters-Wendisch et al. (Microbiology 144, 915-927 (1998)) to switch off the pyc gene of C. glutamicum by a deletion.

A deletion, insertion or a base replacement can be incorporated into the sugA gene in this way.

In addition, it may be advantageous for the production of L-amino acids, as well as attenuating the sugA gene, also to enhance, in particular overexpress, one or more enzymes of the respective biosynthesis pathway, namely glycolysis, anaplerosis, citric acid cycle, pentose phosphate cycle, amino acid export and, optionally, regulatory proteins.

The term “enhancement” describes in this connection the raising of the intracellular activity of one or more enzymes (proteins) in a microorganism that are coded by the corresponding DNA, by for example increasing the number of copies of the gene or genes, using a strong promoter, or using a gene or allele that codes for a corresponding enzyme (protein) having a high activity, and optionally combining these measures.

By such enhancement measures, in particular overexpression, the activity or concentration of the corresponding protein is in general raised by at least 10%, 25%, 50%, 75%, 100%, 150%, 200%, 300%, 400% or 500%, at most up to 1000% or 2000%, referred to that of the wild type protein and/or to the activity or concentration of the protein in the starting microorganism.

Thus for example, for the production of L-amino acids, in addition to the attenuation of the sugA gene one or more of the genes selected from the following group may at the same time be enhanced, in particular overexpressed:

the gene dapA coding for dihydrodipicolinate synthase (EP-B 0 197 335),

the gene gap coding for glyceraldehyde-3-phosphate dehydrogenase (Eikmanns (1992), Journal of Bacteriology 174:6076-6086),

the gene tpi coding for triosephosphate isomerase (Eikmanns (1992), Journal of Bacteriology 174:6076-6086),

the gene pgk coding for 3-phosphoglycerate kinase (Eikmanns (1992), Journal of Bacteriology 174:6076-6086),

the gene zwf coding for glucose-6-phosphate dehydrogenase (JP-A-09224661),

the gene pyc coding for pyruvate carboxylase (DE-A-198 31 609),

the gene mqo coding for malate-quinone-oxidoreductase (Molenaar et al., European Journal of Biochemistry 254, 395-403 (1998)),

the gene lysC coding for a feedback-resistant aspartate kinase (EP-B-0387527; EP-A-0699759; WO 00/63388),

the gene lysE coding for lysine export (DE-A-195 48 222),

the gene hom coding for homoserine dehydrogenase (EP-A 0131171),

the gene ilvA coding for threonine dehydratase (Möckel et al., Journal of Bacteriology (1992) 8065-8072)) or the allele ilvA(Fbr) coding for a feedback-resistant threonine dehydratase (Möckel et al., (1994) Molecular Microbiology 13: 833-842),

the gene ilvBN coding for acetohydroxy acid synthase (EP-B 0356739),

the gene ilvD coding for dihydroxy acid dehydratase (Sahm and Eggeling (1999) Applied and Environmental Microbiology 65: 1973-1979),

the gene zwa1 coding for the Zwa1 protein (DE: 19959328.0, DSM 13115).

Furthermore, it may be advantageous for the production of amino acids, in addition to the attenuation of the sugA gene, also at the same time to attenuate, in particular to reduce the expression, of one or more genes selected from the group

the gene pck coding for phosphoenol pyruvate carboxykinase (DE 199 50 409.1, DSM 13047),

the gene pgi coding for glucose-6-phosphate isomerase (U.S. Ser. No. 09/396,478, DSM 12969),

the gene poxB coding for pyruvate oxidase (DE: 1995 1975.7, DSM 13114),

the gene zwa2 coding for the Zwa2 protein (DE: 19959327.2, DSM 13113)

In addition, it may be advantageous for the production of amino acids, in addition to the attenuation of the sugA gene also to switch off undesirable secondary reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms produced according to the invention are likewise the subject of the invention and may be cultivated continuously or batchwise in a batch process (batch cultivation) or in a fed batch process (feed process) or repeated fed batch process (repetitive feed process) for the purposes of production of L-amino acids. A summary of know cultivation methods is given in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Brunswick/Wiesbaden, 1994)).

The culture medium to be used must suitably satisfy the requirements of the relevant strains. Descriptions of culture media for various microorganisms are given in the manual “Manual of Methods for General Bacteriology” of the American Society for Bacteriology (Washington D.C., USA, 1981).

Carbon sources that may be used include sugars and carbohydrates such as for example glucose, sucrose, lactose, fructose, maltose, molasses, starch and cellulose, oils and fats such as for example soya bean oil, sunflower oil, peanut oil and coconut oil, fatty acids such as for example palmitic acid, stearic acid and linoleic acid, alcohols such as for example glycerol and ethanol, and organic acids such as for example acetic acid. These substances may be used individually or as a mixture.

Nitrogen sources that may be used include organic nitrogen-containing compounds such as peptones, yeast extract, meat extract, malt extract, corn steep liquor, soya bean flour and urea, or inorganic compounds such as ammonium sulfate, ammonium chloride, ammonium phosphate, ammonium carbonate and ammonium nitrate. The nitrogen sources may be used individually or as a mixture.

Phosphorus sources that may be used include phosphoric acid, potassium dihydrogen phosphate or dipotassium hydrogen phosphate or the corresponding sodium salts. The culture medium must furthermore contain salts of metals, such as for example magnesium sulfate or iron sulfate, that are necessary for growth. Finally, essential growth promoters such as amino acids and vitamins may be used in addition to the aforementioned substances. Suitable precursors may furthermore be added to the culture medium. The aforementioned starting substances may be added to the culture in the form of a single one-off batch, or may be suitably metered in during the culture process.

Basic compounds such as sodium hydroxide, potassium hydroxide, ammonia or ammonia water, or acidic compounds such as phosphoric acid or sulfuric acid, are used in a suitable manner in order to control the pH of the culture. Anti-foaming agents such as for example fatty acid polyglycol esters may be used to control foam formation. In order to maintain the stability of plasmids suitable selectively acting substances such as for example antibiotics may be added to the medium. In order to maintain aerobic conditions, oxygen or oxygen-containing gas mixtures such as for example air are introduced into the culture. The temperature of the culture is normally 20° C. to 45° C. and preferably 25° C. to 40° C. The culture is continued until a maximum of the desired product has been formed. This objective is normally achieved within 10 hours to 160 hours.

Methods for the determination of L-amino acids are known. The analysis may be carried out for example as described by Spackman et al. (Analytical Chemistry, 30, (1958), 1190) by ion exchange chromatography followed by ninhydrin derivation, or can be carried out by reversed phase HPLC, as described by Lindroth et al. (Analytical Chemistry (1979) 51: 1167-1174).

The process according to the invention provides for the enzymatic production of amino acids.

The following microorganism was deposited on 12.01.2001 as a pure culture at the German Collection of Microorganisms and Cell Cultures (DSMZ, Brunswick, Germany) according to the Budapest Convention:

Escherichia coli strain Top10/pCR2.1sugAint as DSM 13986.

### EXAMPLES

Having generally described this invention, a further understanding can be obtained by reference to certain specific examples which are provided herein for purposes of illustration only and are not intended to be limiting unless otherwise specified.

The isolation of plasmid DNA from Escherichia coli as well as all techniques involved in restriction, Klenow treatment and alkaline phosphatase treatment have been carried out by Sambrook et al. (Molecular Cloning. A Laboratory Manual, 1989, Cold Spring Harbor Laboratory Press, Cold Spring Harbor, N.Y., USA). Methods for the transformation of Escherichia coli are also described in that manual.

The composition of readily available nutrient media such as LB or TY media are also described by Sambrook et al.

### Example 1

### Production of a Genomic Cosmid Gene Library from C. glutamicum ATCC 13032

Chromosomal DNA from C. glutamicum ATCC 13032 was isolated as described by Tauch et al. (1995, Plasmid 33:168-179) and partially cleaved with the restriction enzyme Sau3AI (Amersham Pharmacia, Freiburg, Germany, product description Sau3AI, Code no. 27-0913-02). The DNA fragments were desphosphorylated with shrimp alkaline phosphatase (Roche Molecular Biochemicals, Mannheim, Germany, product description SAP, Code no. 1758250). The DNA of the cosmid vector SuperCos1 (Wahl et al. (1987) Proceedings of the National Academy of Sciences USA 84:2160-2164), obtained from Stratagene (La Jolla, USA, product description SuperCos1 Cosmid Vector Kit, Code no. 251301) was cleaved with the restriction enzyme XbaI (Amersham Pharmacia, Freiburg, Germany, product description XbaI, Code no. 27-0948-02) and likewise dephosphorylated with shrimp alkaline phosphatase.

The cosmid DNA was then cleaved with the restriction enzyme BamHI (Amersham Pharmacia, Freiburg, Germany, product description BamHI, Code no. 27-0868-04). The cosmid DNA treated in this way was mixed with the treated ATCC13032-DNA and the batch was treated with T4-DNA ligase (Amersham Pharmacia, Freiburg, Germany, product description T4-DN ligase, Code no. 27-0870-04). The ligation mixture was then packed into phages using the Gigapack II XL Packing Extracts (Stratagene, La Jolla, USA, product description Gigapack II XL Packing Extract, Code no. 200217).

For the infection of the E. coli strain NM554 (Raleigh et al. 1988, Nucleic Acid Research 16:1563-1575) the cells were taken up in 10 mM MgSO₄ and mixed with an aliquot of the phage suspension. Infection and titration of the cosmid library were carried out as described by Sambrook et al. (1989, Molecular Cloning: A Laboratory Manual, Cold Spring Harbor), the cells having been plated out on LB agar (Lennox, 1955, Virology, 1:190) with 100 μg/ml ampicillin. Recombinant individual clones were selected after incubation overnight at 37° C.

### Example 2

### Isolation and Sequencing of the sugA Gene

The cosmid DNA of an individual colony was isolated using the Qiaprep Spin Miniprep Kit (Product No. 27106, Qiagen, Hilden, Germany) according to the manufacturer's instructions and partially cleaved with the restriction enzyme Sau3AI (Amersham Pharmacia, Freiburg, Germany, product description Sau3AI, Product No. 27-0913-02). The DNA fragments were dephosphorylated with shrimp alkaline phosphatase (Roche Molecular Biochemicals, Mannheim, Germany, product description SAP, Product No. 1758250). After gel electrophoresis separation, the cosmid fragments were isolated in an order of magnitude of 1500 to 2000 bp using the QiaExII Gel Extraction Kit (Product No. 20021, Qiagen, Hilden, Germany).

The DNA of the sequencing vector pZero-1, obtained from Invitrogen (Groningen, Netherlands, product description Zero Background Cloning Kit, Product No. K2500-01), was cleaved with the restriction enzyme BamHI (Amersham Pharmacia, Freiburg, Germany, product description BamHI, Product No. 27-0868-04). The ligation of the cosmid fragments in the sequencing vector pZero-1 was carried out as described by Sambrook et al. (1989, Molecular Cloning: A Laboratory Manual, Cold Spring Harbor), the DNA mixture having been incubated overnight with T4 ligase (Pharmacia Biotech, Freiburg, Germany). This ligation mixture was then electroporated (Tauch et al. 1994, FEMS Microbiol. Letters, 123:343-7) into the E. coli strain DH5aMCR (Grant, 1990, Proceedings of the National Academy of Sciences, U.S.A., 87:4645-4649) and plated out onto LB agar (Lennox, 1955, Virology, 1:190) with 50 μg/ml zeocin.

The plasmid preparation of the recombinant clone was performed with the Biorobot 9600 (Product No. 900200, Qiagen, Hilden, Germany). The sequencing was carried out according to the dideoxy chain termination method of Sanger et al. (1977, Proceedings of the National Academy of Sciences U.S.A., 74:5463-5467) as modified by Zimmermann et al. (1990, Nucleic Acids Research, 18:1067). The “RR dRhodamin Terminator Cycle Sequencing Kit” of PE Applied Biosystems (Product No. 403044, Weiterstadt, Germany) was used. The gel electrophoresis separation and analysis of the sequencing reaction was carried out in a “rotiphoresis NF acrylamide/bisacrylamide” gel (29:1) (Product No. A124.1, Roth, Karlsruhe, Germany) using the “ABI Prism 377” sequencing apparatus from PE Applied Biosystems (Weiterstadt, Germany).

The raw sequencing data obtained were then processed using the Staden program package (1986, Nucleic Acids Research, 14:217-231) Version 97-0. The individual sequences of the pZero1 derivatives were assembled into a coherent contig. The computer-assisted coding region analysis was prepared using the XNIP program (Staden, 1986, Nucleic Acids Research, 14:217-231). Further analyses were carried out with the “BLAST search programs” (Altschul et al., 1997, Nucleic Acids Research, 25:33893402) against the non-redundant databank of the “National Center for Biotechnology Information” (NCBI, Bethesda, Md., USA).

The nucleotide sequence obtained is shown in SEQ ID No. 1. The analysis of the nucleotide sequence revealed an open reading frame of 1035 base pairs, which was termed the sugA gene. The sugA gene codes for a polypeptide of 344 amino acids.

### Example 3

### Production of an Integration Vector for the Integration Mutagenesis of the sugA Gene

Chromosomal DNA was isolated from the strain ATCC 13032 by the method of Eikmanns et al. (Microbiology 140: 1817-1828 (1994)). On the basis of the sequence of the sugA gene known from Example 2 for C. glutamicum, the following oligonucleotides were selected for the polymerase chain reaction (see SEQ ID No. 3 and SEQ ID No. 4):

sugA-int1:
5′ AGC GAT TCT TAT CCC TTG G 3′
sugA-int2:
5′AGC AGG AAG ATC AGT GTG G 3′

The illustrated primers were synthesized by MWG-Biotech AG (Ebersberg, Germany) and the PCR reaction was carried out according to the standard PCR method of Innis et al. (PCR Protocols. A Guide to Methods and Applications, 1990, Academic Press) using Taq DNA polymerase from Boehringer Mannheim, (Germany, product description Taq DNA Polymerase, Product No. 1 146 165). By means of the polymerase chain reaction the primers permit the amplification of a 483 bp long internal fragment of the sugA gene. The thus amplified product was electrophoretically tested in a 0.8% agarose gel.

The amplified DNA fragment was ligated into the vector pCR2.1-TOPO (Mead at al. (1991) Bio/Technology 9:657-663) using the TOPO TA Cloning Kit from Invitrogen Corporation (Carlsbad, Calif., USA; Cat. No. K4500-01).

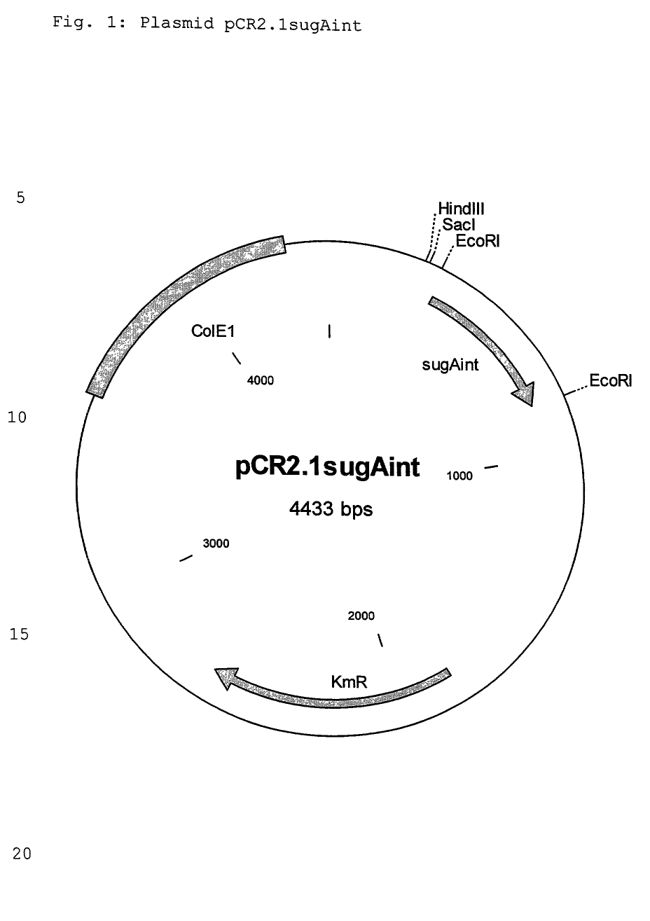

The E. coli strain TOP10 was then electroporated with the ligation batch (Hanahan, In: DNA Cloning. A Practical Approach. Vol. I, IRL-Press, Oxford, Washington D.C., USA, 1985). Plasmid-carrying cells were selected by plating out the transformation batch onto LB agar (Sambrook et al., Molecular Cloning: A Laboratory Manual. 2^{nd} Ed., Cold Spring Harbor Laboratory Press, Cold Spring Harbor, N.Y., 1989) that had been supplemented with 50 mg/l of kanamycin. Plasmid DNA was isolated from a transformant using the QIAprep Spin Miniprep Kit from Qiagen and was checked by restriction with the restriction enzyme EcoRI followed by agarose gel electrophoresis (0.8%). The plasmid was named pCR2.1 sugAint and is shown in FIG. 1.

### Example 4

### Integration Mutagenesis of the sugA Gene in the Strain DSM 5715

The vector pCR2.1 sugAint mentioned in Example 3 was electroporated into Corynebacterium glutamicum DSM 5715 according to the electroporation method of Tauch et. al. (FEMS Microbiological Letters, 123:343-347 (1994)). The strain DSM 5715 is an AEC-resistant lysine producer. The vector pCR2.1 sugAint cannot replicate independently in DSM 5715 and thus only remains in the cell if it has integrated into the chromosome of DSM 5715. The selection of clones with pCR2.1 sugAint integrated into the chromosome was made by plating out the electroporation batch onto LB agar (Sambrook et al., Molecular Cloning: A Laboratory Manual. 2^{nd} Ed., Cold Spring Harbor Laboratory Press, Cold Spring Harbor, N.Y.) that had been supplemented with 15 mg/l of kanamycin.

In order to demonstrate the integration the sugAint fragment was labeled using the Dig Hybridization Kit from Boehringer according to the method described in “The DIG System User's Guide for Filter Hybridization” published by Boehringer Mannheim GmbH (Mannheim, Germany, 1993). Chromosomal DNA of a potential integrant was isolated according to the method of Eikmanns et al. (Microbiology 140: 1817-1828 (1994)) and was in each case cleaved with the restriction enzymes SacI, EcoRI and HindIII. The resultant fragments were separated by means of agarose gel electrophoresis and hybridized at 68° C. using the Dig Hybridization Kit from Boehringer. The plasmid pCR2.1 sugAint mentioned in Example 3 had inserted itself within the chromosomal sugA gene into the chromosome of DSM 5715. The strain was designated DSM5715::pCR2.1sug-Aint.

### Example 5

### Production of Lysine

The C. glutamicum strain DSM5715::pCR2.1sugAint obtained in Example 4 was cultivated in a nutrient medium suitable for the production of lysine and the lysine content in the culture supernatant was determined.

For this purpose the strain was first of all incubated for 24 hours at 33° C. on an agar plate with the corresponding antibiotic (brain-heart agar with kanamycin (25 mg/l). Starting from this agar plate culture a preculture was inoculated (10 ml of medium in a 100 ml Erlenmeyer flask). The full medium CgIII was used as medium for the preculture.

Medium Cg III

| | | |
|---|---|---|
| NaCl | 2.5 | g/l |
| Bacto-Peptone | 10 | g/l |
| Bacto-Yeast Extract | 10 | g/l |
| Glucose (autoclaved separately) | 2% | (w/v) |
| The pH value was adjusted to pH 7.4 | | |

Kanamycin (25 mg/l) was added to this preculture. The preculture was then incubated for 16 hours at 33° C. at 240 rpm on a shaker table. From this preculture a main culture was inoculated so that the initial OD (660 nm) of the main culture was 0.1 OD. The medium MM was used for the main culture.

| | Medium MM | |
|---|---|---|
| CSL (Corn Steep Liquor | 5 | g/l |
| MOPS | 20 | g/l |
| Glucose (autoclaved separately) | 50 | g/l |
| Salts: | | |
| (NH₄)₂SO₄ | 25 | g/l |
| KH₂PO₄ | 0.1 | g/l |
| MgSO₄.7H₂O | 1.0 | g/l |
| CaCl₂ .2H₂ | 10 | mg/l |
| FeSO₄.7H₂O | 10 | mg/l |
| MnSO₄.H₂ | 5.0 | mg/l |
| Biotin (sterile filtered) | 0.3 | mg/l |
| Thiamine · HCl (sterile filtered) | 0.2 | mg/l |
| Leucine (sterile filtered) | 0.1 | g/l |
| CaCO₃ | 25 | g/l |

CSL, MOPS and the salt solution are adjusted with ammonia water to pH 7 and autoclaved. The sterile substrate solutions and vitamin solutions as well as the dry autoclaved CaCO₃ are then added.

Cultivation is carried out in a 10 ml volume in a 100 ml Erlenmeyer flask equipped with baffles. Kanamycin was added (25 mg/l). The cultivation was carried out at 33° C. and 80% atmospheric humidity.

After 72 hours the OD was determined at a measurement wavelength of 660 nm with a Biomek 1000 (Beckmann Instruments GmbH, Munich). The amount of lysine formed was determined by ion exchange chromatography and post-column derivation with ninhydrin detection using an amino acid analyzer from Eppendorf-BioTronik (Hamburg, Germany).

The results of the experiment are shown in Table 1.

TABLE 1

| | OD | Lysine-HCl |
|---|---|---|
| Strain | (660 nm) | g/l |
| DSM5715 | 8.2 | 13.74 |
| DSM5715::pCR2.1sugAint | 9.2 | 14.17 |

All of the publications cited above are incorporated herein by reference.

Obviously, numerous modifications and variations of the present invention are possible in light of the above teachings. It is therefore to be understood that within the scope of the appended claims, the invention may be practiced otherwise than as specifically described herein.

This application is based on German Patent Application Serial No. 100 45 485.2, filed on Sep. 14, 2000, and German Patent Application Serial No. 101 08 839.6 filed on Feb. 23, 2001, both of which are incorporated herein by reference.

What is claimed is:

1. An isolated polynucleotide from coryneform bacteria containing a polynucleotide sequence coding for the sugA gene, selected from the group consisting of
(a) a polynucleotide that is at least 70% identical to a polynucleotide coding for a polypeptide that contains the amino acid sequence of SEQ ID No. 2,
(b) a polynucleotide coding for a polypeptide that contains an amino acid sequence that is at least 70% identical to the amino acid sequence of SEQ ID No. 2,
(c) a polynucleotide that is complementary to the polynucleotides of (a) or (b), and
(d) a polynucleotide containing at least at least 15 successive nucleotides of the polynucleotide sequence of (a), (b), or (c),

2. The isolated polynucleotide of claim 1, wherein the polypeptide has the activity of the sugar transport protein SugA.

3. The isolated polynucleotide of claim 1, which is replicable in coryneform bacteria.

4. The isolated polynucleotide of claim 1, which is a recombinant DNA replicable in coryneform bacteria.

5. The isolated polynucleotide of claim 1, wherein the polynucleotide is an RNA.

6. The isolated polynucleotide of claim 1, containing the nucleic acid sequence as shown in SEQ ID No. 1.

7. The isolated polynucleotide of claim 3, containing
(i) the nucleotide sequence shown in SEQ ID No. 1, or
(ii) at least one sequence that corresponds to the sequence (i) within the region of degeneracy of the genetic code, or
(iii) at least one sequence that hybridizes with the sequence that is complementary to the sequence (i) or (ii), and, optionally,
(iv) functionally neutral sense mutations in (i).

8. The isolated polynucleotide of claim 7, wherein the hybridization is carried out under conditions of stringency corresponding at most to 2× SSC.

9. The isolated polynucleotide of claim 1, that codes for a polypeptide that contains the amino acid sequence shown in SEQ ID No. 2.

10. The isolated polynucleotide of claim 1, which is (a).

11. The isolated polynucleotide of claim 1, which is (b).

12. The isolated polynucleotide of claim 1, which is (c).

13. The isolated polynucleotide of claim 1, which is (d).

14. The vector pCR2.1sugAint having the restriction map shown in FIG. 1.

15. The vector of claim 14, which has been introduced in the E. coli strain Top10/pCR2.1sugAint under No. DSM 13986 at the German Collection for Microorganisms and Cell Cultures.

16. A vector which carries a 483 bp long internal fragment of the sugA gene.

17. The vector of claim 16, which has been introduced in the E. coli strain Top10pCR2.1sugAint under No. DSM 13986 at the German Collection for Microorganisms and Cell Cultures.

18. An internal fragment of the sugA gene having a length of 483 bp.

19. Coryneform bacteria, in which the sugA gene is attenuated.

20. The Coryneform bacteria of claim 19, in which the sugA gene is switched off.

21. A process for the enzymatic production of an L-amino acid, comprising:
(a) fermentating coryneform bacteria producing the L-amino acid in a medium, wherein at least the sugA gene or a nucleotide sequence coding for the latter is attenuated in the bacteria,
(b) enriching the amount of the L-amino acid in the medium or in the cells of the bacteria, and
(c) isolating the L-amino acid.

22. The process of claim 21, wherein at least the sugA gene or a nucleotide sequence coding for the latter is switched off.

23. The process of claim 21, wherein the amino acid is L-lysine.

24. The process of claim 21, wherein the amino acid is selected from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-tryptophan, L-arginine, and salts thereof.

25. The process of claim 21, wherein additional genes of the biosynthesis pathway of the L-amino acid are enhanced in the bacteria.

26. The process of claim 21, wherein the metabolic pathways that reduce the formation of the L-amino acid are at least partially switched off in the bacteria.

27. The process of claim 21, wherein the expression of the polynucleotide(s) that code(s) for the sugA gene is attenuated.

28. The process of claim 21, wherein the expression of the polynucleotide(s) that code(s) for the sugA gene is switched off.

29. The process of claim 21, wherein the catalytic properties of the polypeptide that codes for the polynucleotide sugA are reduced.

30. The process of claim 21, wherein at the same time one or more of the genes selected from group consisting of
the gene dapA coding for dihydrodipicolinate synthase,
the gene gap coding for glyceraldehyde-3-phosphate dehydrogenase,
the gene tpi coding for triosephosphate isomerase,
the gene pgk coding for 3-phosphoglycerate kinase,
the gene zwf coding for glucose-6-phosphate dehydrogenase,
the gene pyc coding for pyruvate carboxylase, the gene mqo coding for malate-quinone-oxidoreductase,
the gene lysC coding for a feedback-resistant aspartate kinase,
the gene lyse coding for lysine export,
the gene hom coding for homoserine dehydrogenase,
the gene ilvA coding for threonine dehydratase or the allele ilvA(Fbr) coding for a feedback-resistant threonine dehydratase,
the gene ilvBN coding for acetohydroxy acid synthase,
the gene ilvD coding for dihydroxy acid dehydratase, and
the gene zwa1 coding for the Zwa1 protein, is/are enhanced or overexpressed

31. The process of claim 21, wherein at the same time one or more of the genes selected from group consisting of
the gene pck coding for phosphoenol pyruvate carboxykinase,
the gene pgi coding for glucose-6-phosphate isomerase,
the gene poxB coding for pyruvate oxidase, and
the gene zwa2 coding for the Zwa2 protein.
is/are attenuated.

32. The process of claim 21, wherein bacteria are of the species Corynebacterium glutamicum.

33. Coryneform bacteria that contain a vector that carries parts of the polynucleotide according to claim 1.

34. A process for identifying nucleic acids which code for the sugar transport protein sugA or that have a high degree of similarity to the sequence of the sugA gene, comprising:
contacting a sample with the isolated polynucleotide of claim 1 under conditions suitable for the polynucleotide to hybridize to other nucleic acids which code for the sugar transport protein sugA or that have a high degree of similarity to the sequence of the sugA gene.

35. The process of claim 24, which is conducted on an array, microarray, or a DNA chip.
